(19) United States
(12) Patent Application Publication
(10) Pub. No.: US 2019/0321365 A1
(43) Pub. Date: Oct. 24, 2019

(54) Maintenance Therapy for the Treatment of Cancer

(71) Applicant: Aslan Pharmaceuticals Pte. Ltd., Singapore (SG)

(72) Inventor: Bertil Lindmark, Singapore (SG)

(73) Assignee: Aslan Pharmaceuticals Pte. Ltd., Singapore (SG)

(21) Appl. No.: 16/312,816

(22) PCT Filed: Jun. 30, 2017
(86) PCT No.: PCT/SG2017/050333

§ 371 Date: Dec. 21, 2018

(30) Foreign Application Priority Data
Jul. 1, 2016 (GB) 1611580.0

(51) Int. Cl. A61K 31/517; A61K 9/00

(52) U.S. Cl. CPC A61K 31/517; A61K 9/0053

(57) ABSTRACT

The present disclosure relates to a maintenance therapy for prolongation of remission of a cancer comprising administering a therapeutically effective amount of a compound of formula (I) an enantiomer thereof or pharmaceutically acceptable salt of any one of the same or a pharmaceutical composition thereof.

The present disclosure relates to a maintenance therapy for prolongation of remission of a cancer.

### BACKGROUND

There are many cancers that are difficult to treat and although therapy is available, there appears to exist or to come into existence, a certain degree of resistance to the therapy. Primary resistance may occur, and secondary as well, which means that a therapy to which the patient seems to respond, at some time point loses its efficacy.

Primary cancer therapy includes surgery, chemotherapy, radiotherapy, immunotherapy and combinations thereof. Once primary therapy is completed and the cancer is stable or in remission then the patient may be given a maintenance therapy.

Examples of maintenance therapy include tamoxifen (nolvadex) for ER+(estrogen receptor positive) breast cancer patients, and combination therapies of tamoxifen and bevacizumab (Roviello et al Cancer Biol Ther. 2015 April; 16(4): 493-497—Five years of stable disease with maintenance therapy using bevacizumab and tamoxifen in a patient with metastatic breast cancer).

Tamoxifen is used in certain patient populations that have had breast cancer to reduce the risk of recurrence of the of the caner and prolong the period of cancer remission. “The International Breast Cancer Intervention Study-I was designed to investigate the long-term risks and benefits of taking tamoxifen to prevent breast cancer in women at high risk for developing the disease”. This long-term study showed that tamoxifen reduced the risk of breast cancer by 29%.

The researchers looked at tamoxifen's effects on the risk of specific types of breast cancer:
tamoxifen reduced the risk of estrogen-receptor-positive breast cancer by 35%
tamoxifen didn't reduce the risk of estrogen-receptor-negative breast cancer

The researchers also looked to see if tamoxifen offered more benefits for women who didn't take hormone replacement therapy:
tamoxifen reduced the risk of breast cancer by 38% in women who didn't take hormone replacement therapy
tamoxifen reduced the risk of estrogen-receptor-positive disease by 45% in women who didn't take hormone replacement therapy.

Thus whilst tamoxifen is useful in the particular patient population it would be useful to have maintenance therapies available for other cancers, other patient populations or simply as an alternative to tamoxifen.

Effective maintenance therapies in other cancers and cancer patient populations are still required. The present inventors have established that varlitinib is suitable for use as a cancer maintenance therapy and is well tolerated.

Varlitinib, which has the chemical name (R)-N4-[3-Chloro-4-(thiazol-2-ylmethoxy)-phenyl]-N6-(4-methyl-4,5,-dihydro-oxazol-2-yl)-quinazoline-4,6-diamine (ASLAN001 Example 52 disclosed in WO2005/016346), is a small-molecule pan-HER inhibitor. It has been tested as a monotherapy in phase I clinical trials of gastric cancer patients. 23 patients, who had previously failed on one or more rounds of chemotherapy and, where eligible, trastuzumab, each received 500 mg of ASLAN001 orally twice daily as monotherapy for 28 days. Tumor biopsies taken before and after treatment were analysed using immunohistochemistry. Signs of clinical activity included downregulation of signalling pathways responsible for cell proliferation, and a reduction in cell survival and cell proliferation in gastric tumors that were either co-expressing EGFR and HER2 or that were HER2 amplified.

The anti-cancer activity of Varlitinib which has been demonstrated to date is encouraging and trials are under way to further illustrate its clinical efficacy.

Vartlitinib is a very well tolerated medicament and in comparison to many anti-cancer treatments has very few side effects.

### SUMMARY OF THE DISCLOSURE

The present disclosure will now be summarised in the paragraphs below. Thus there is provided.
1. A method of cancer maintenance therapy comprising administering a therapeutically effective amount of a compound of formula (I):

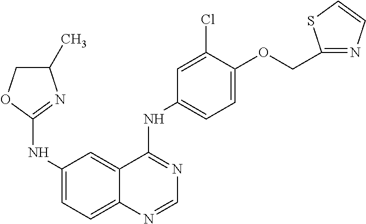 [SMILES: CC1COC(Nc2ccc3ncnc(Nc4ccc(OCc5nccs5)c(Cl)c4)c3c2)=N1]

an enantiomer thereof or pharmaceutically acceptable salt of any one of the same.
2. A method according to paragraph 1, wherein the compound of formula (I) is:

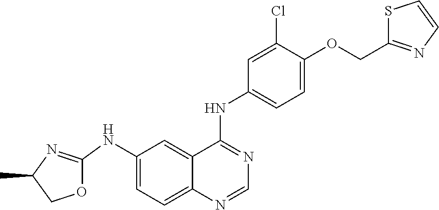 [SMILES: C[C@@H]1COC(Nc2ccc3ncnc(Nc4ccc(OCc5nccs5)c(Cl)c4)c3c2)=N1]

or a pharmaceutically acceptable salt thereof.
3. A method according to paragraph 1 or 2, wherein the compound of formula (I) is provided as the free base.
4. A method according to any one of paragraphs 1 to 3, wherein the therapy is continues for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 months or more.
5. A method according to any one of paragraphs 1 to 4, wherein the cancer is an epithelial cancer.
6. A method according to any one of paragraphs 1 to 5, wherein the cancer is selected from the comprising liver cancer, biliary tract cancer, breast cancer (such as none ER+breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer.
7. A method according to paragraph 6, wherein the cancer is selected from gastric cancer, hepatocellular carcinoma and cholangiocarcinoma.
8. A method according to any one of paragraphs 1 to 7, wherein the cancer is HER2 positive or HER2 amplified.
9. A method according to any one of paragraphs 1 to 8, wherein each dose of the compound of formula (I) or composition comprising the same is in the range 100 to 900 mg.
10. A method according to paragraph 9, wherein each dose is in the range of 300 to 500 mg, such as 400 mg.
11. A method according to any one of paragraphs 1 to 10, wherein the compound of formula (I) or a pharmaceutical composition comprising the same is administered orally.
12. A method according to any one of paragraphs 1 to 11, wherein the compound of formula (I) or a pharmaceutical formulation thereof is administered once daily or bi-daily, such as bi-daily
13. A method according to any one of paragraphs 1 to 12, wherein the period of cancer remission is prolonged in comparison to a patient population which do not receive a maintenance therapy.
14. A method according to any one of paragraphs 1 to 13, wherein the remission is full remission.
15. A method according to any one of paragraphs 1 to 13, wherein the remission is partial remission.
16. A method according to any one of paragraphs 1 to 13, wherein the patient's life expectancy is increased in comparison to a patient population which do not receive a maintenance therapy.
17. A method according to any one of paragraphs 1 to 16, wherein the patient's creatinine levels (in particular G3 creatinine levels) are monitored during the period of maintenance therapy.
18. A method according to any one of paragraphs 1 to 17, wherein the dose of the compound of formula (I) is reduced in the treatment period, for example to be in the range 100 to 300 mg (such as 200 mg) administered once or twice daily.
19. A method according to paragraph 18, wherein the dose reduction is response to an increase in creatinine levels (in particular an increase in G3 creatinine levels).
20. A compound of formula (I):

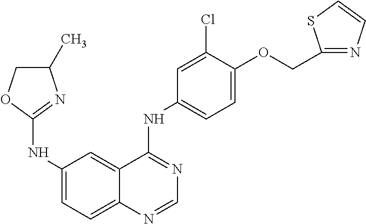 [SMILES: CC1COC(Nc2ccc3ncnc(Nc4ccc(OCc5nccs5)c(Cl)c4)c3c2)=N1]

an enantiomer thereof, a pharmaceutically acceptable salt thereof or a composition comprising any one of the same for use in a cancer maintenance therapy.
21. A compound for use according to paragraph 20, wherein the compound of formula (I) is:

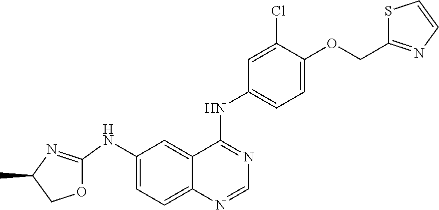 [SMILES: C[C@@H]1COC(Nc2ccc3ncnc(Nc4ccc(OCc5nccs5)c(Cl)c4)c3c2)=N1]

or a pharmaceutically acceptable salt thereof.
22. A compound for use according to paragraph 20 or 21, wherein the compound of formula (I) is provided as the free base.
23. A compound for use according to any one of paragraphs 20 to 22, wherein the therapy is continues for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 months or more.
24. A compound for use according to any one of paragraphs 20 to 23, wherein the cancer is an epithelial cancer.
25. A compound for use according to any one of paragraphs 20 to 24, wherein the cancer is selected from the comprising liver cancer, biliary tract cancer, breast cancer (such as none ER+breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer.
26. A compound for use according to paragraph 25, wherein the cancer is selected from gastric cancer, hepatocellular carcinoma and cholangiocarcinoma.
27. A compound for use according to any one of paragraphs 20 to 26, wherein the cancer is HER2 positive or HER2 amplified.
28. A compound for use according to any one of paragraphs 20 to 27, wherein each dose of the compound of formula (I) or composition comprising the same is in the range 100 to 900 mg.
29. A compound for use according to paragraph 28, wherein each dose is in the range of 300 to 500 mg, such as 300 or 400 mg.
30. A compound of use according to any one of paragraphs 20 to 29, wherein the compound of formula (I) or a pharmaceutical composition comprising the same is administered orally.
31. A compound for use according to any one of paragraphs 20 to 30, wherein the compound of formula (I) or a pharmaceutical formulation thereof is administered once a day or bi-daily, for example bi-daily.
32. A compound for use according to any one of paragraphs 20 to 31, wherein the period of cancer remission is prolonged in comparison to a patient population which do not receive a maintenance therapy.
33. A compound for use according to any one of paragraphs 20 to 32, wherein the remission is full remission.
34. A compound for use according to any one of paragraphs 20 to 33, wherein the remission is partial remission.
35. A compound for use according to any one of paragraphs 20 to 34, wherein the patient's life expectancy is increased in comparison to a patient population which do not receive a maintenance therapy.
36. A compound for use according to any one of paragraphs 20 to 35, wherein the patient's creatinine levels (in particular G3 creatinine levels) are monitored during the period of maintenance therapy.
37. A method according to any one of paragraphs 20 to 36, wherein the dose the compound of formula (I) is reduced in the treatment period, for example to be in the range 100 to 300 mg (such as 200 mg) administered once or twice daily.
38. A compound for use according to claim 37, wherein the dose reduction is response to an increase in creatinine levels (in particular an increase in G3 creatinine levels).
39. Use of a compound of formula (I):

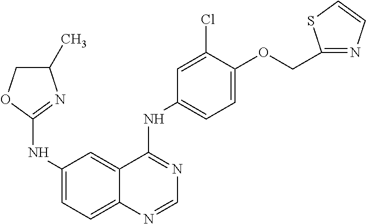 [SMILES: CC1COC(Nc2ccc3ncnc(Nc4ccc(OCc5nccs5)c(Cl)c4)c3c2)=N1]

an enantiomer thereof, a pharmaceutically acceptable salt thereof (or a composition comprising any one of the same) in the manufacture of a medicament for cancer maintenance therapy.
40. Use according to paragraph 39, wherein the compound of formula (I) is:

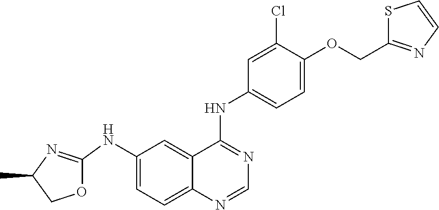 [SMILES: C[C@@H]1COC(Nc2ccc3ncnc(Nc4ccc(OCc5nccs5)c(Cl)c4)c3c2)=N1]

or a pharmaceutically acceptable salt thereof.
41. Use according to paragraph 39 or 40 wherein the compound of formula (I) is provided as the free base.
42. Use according to any one of paragraphs 39 to 41, wherein the therapy is continues for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 months or more.
43. Use according to any one of paragraphs 39 to 42, wherein the cancer is an epithelial cancer.
44. Use according to any one of paragraphs 39 to 43, wherein the cancer is selected from the comprising liver cancer, biliary tract cancer, breast cancer (such as none ER+breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer.
45. Use according to paragraph 44, wherein the cancer is selected from gastric cancer, hepatocellular carcinoma and cholangiocarcinoma.
46. Use according to any one of paragraphs 39 to 45, wherein the cancer is HER2 positive or HER2 amplified.
47. Use according to any one of paragraphs 39 to 46, wherein each dose of the compound of formula (I) or composition comprising the same is in the range 100 to 900 mg.
48. Use according to claim 47, wherein each dose is in the range of 300 to 500 mg, such as 300 or 400 mg.
49. Use according to any one of paragraphs 39 to 48, wherein the compound of formula (I) or a pharmaceutical composition comprising the same is administered orally.
50. Use according to any one of paragraphs 39 to 49, wherein the compound of formula (I) or a pharmaceutical formulation thereof is administered once a day or bi-daily, for example bi-daily.
51. Use according to any one of paragraphs 39 to 50, wherein the period of cancer remission is prolonged in comparison to a patient population which do not receive a maintenance therapy.
52. Use according to any one of paragraphs 39 to 51, wherein the remission is full remission.
53. Use according to any one of paragraphs 39 to 51, wherein the remission is partial remission.
54. Use according to any one of paragraphs 39 to 53, wherein the patient's life expectancy is increased in comparison to a patient population which do not receive a maintenance therapy.
55. Use according to any one of paragraphs 39 to 54, wherein the patient's creatinine levels (in particular G3 creatinine levels) are monitored during the period of maintenance therapy.
56. Use according to any one of paragraphs 39 to 55, wherein the dose the compound of formula (I) is reduced in the treatment period, for example to be in the range 100 to 300 mg (such as 200 mg) administered once or twice daily.
57. Use according to claim 56, wherein the dose reduction is response to an increase in creatinine levels (in particular an increase in G3 creatinine levels).
58. A method of producing a compound of formula (I):

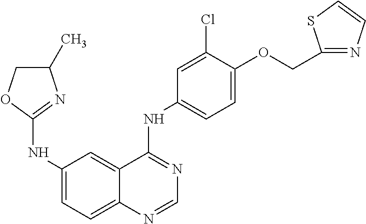 [SMILES: CC1COC(Nc2ccc3ncnc(Nc4ccc(OCc5nccs5)c(Cl)c4)c3c2)=N1]

an enantiomer thereof or pharmaceutically acceptable salt of any one of the same (or a pharmaceutical formulation comprising any one of the same), for use a cancer maintenance therapy.
59. A method according to paragraph 58, wherein the compound of formula (I) is:

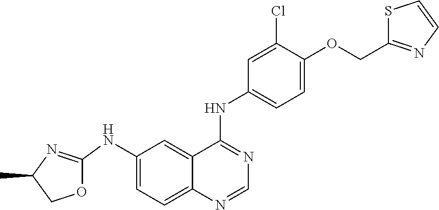 [SMILES: C[C@@H]1COC(Nc2ccc3ncnc(Nc4ccc(OCc5nccs5)c(Cl)c4)c3c2)=N1]

or a pharmaceutically acceptable salt thereof (or a pharmaceutical formulation comprising the same).
60. A method according to paragraph 58 or 59, wherein the compound of formula (I) is provided as the free base.
61. A method according to any one of paragraphs 58 to 60, wherein the therapy is continues for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 months or more.
62. A method according to any one of paragraphs 58 to 61, wherein the cancer is an epithelial cancer.
63. A method according to any one of paragraphs 58 to 62, wherein the cancer is selected from the comprising liver cancer, biliary tract cancer, breast cancer (such as none ER+breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer.
64. A method according to paragraph 63, wherein the cancer is selected from gastric cancer, hepatocellular carcinoma and cholangiocarcinoma.
65. A method according to any one of paragraphs 58 to 64, wherein the cancer is HER2 positive or HER2 amplified.
66. A method according to any one of paragraphs 58 to 65, wherein each dose of the compound of formula (I) or composition comprising the same is in the range 100 to 900 mg.
67. A method according to paragraph 66, wherein each dose is in the range of 300 to 500 mg, such as 400 mg.
68. A method according to any one of paragraphs 58 to 67, wherein the compound of formula (I) or a pharmaceutical composition comprising the same is administered orally.
69. A method according to any one of paragraphs 58 to 68, wherein the compound of formula (I) or a pharmaceutical formulation thereof is administered once daily or bi-daily, such as bi-daily 70. A method according to any one of paragraphs 58 to 69, wherein the period of cancer remission is prolonged in comparison to a patient population which do not receive a maintenance therapy.
71. A method according to any one of paragraphs 58 to 70, wherein the remission is full remission.
72. A method according to any one of paragraphs 58 to 71, wherein the remission is partial remission.
73. A method according to any one of paragraphs 58 to 72, wherein the patient's life expectancy is increased in comparison to a patient population which do not receive a maintenance therapy.
74. A method according to any one of paragraphs 58 to 73, wherein the patient's creatinine levels (in particular G3 creatinine levels) are monitored during the period of maintenance therapy.
75. A method according to any one of paragraphs 58 to 74, wherein the dose of the compound of formula (I) is reduced in the treatment period, for example to be in the range 100 to 300 mg (such as 200 mg) administered once or twice daily.
76. A method according to claim 75, wherein the dose reduction is response to an increase in creatinine levels (in particular an increase in G3 creatinine levels).

In one embodiment the cancer is an epithelial cancer, for example a carcinoma.

In one embodiment the cancer is selected from the group comprising liver cancer, biliary tract cancer, breast cancer (such as none ER+breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer, for example the cancer is selected from gastric cancer, hepatocellular carcinoma and cholangiocarcinoma.

In one embodiment the liver cancer is primary liver cancer. In one embodiment the liver cancer is secondary liver cancer. In one embodiment the liver cancer is stage 1, 2, 3A, 3B, 3C, 4A or 4B.

In one embodiment the gastric cancer is stage 0, I, II, III or IV.

In one embodiment the cancer is a tumour, such as a solid tumour.

In one embodiment the treatment according to the present disclosure is suitable for administration to a patient with secondary tumours. In one embodiment the patient receiving the therapy of the present disclosure has metastatic cancer.

In one embodiment the therapy according to the present disclosure is suitable for the treatment of a patient with primary cancer and metastases.

In one embodiment the therapy according to the present disclosure is suitable for administration to a patient with cancerous cells in a lymph node.

In one embodiment the cancer is HER2 positive or HER2 amplified.

In one embodiment the cancer overexpresses at least one HER receptor, for example HER1, HER2, HER3, HER4 and combinations thereof, for example 2, 3 or 4 of said receptors.

In one embodiment the patient population is HER 1 positive.

In one embodiment the patient population is HER 2 positive.

In one embodiment the patient population is HER 3 positive.

In one embodiment the patient population is HER 4 positive.

In one embodiment the target patient population is EGFR and HER2 positive or are HER2 amplified.

In one embodiment the patient population for treatment has HER pathway activation, for example indicated by high levels of phosphorylated HER receptor or HER receptors, such as receptors selected from HER1, HER2, HER3 and HER4.

In one embodiment, the patient population for treatment has HER pathway activation indicated by high levels of phosphorylated downstream signalling proteins, for example selected from pAKT and pERK.

In one embodiment the maintenance therapy of the present disclosure is given to a patient in remission, in particular full remission.

In one embodiment the maintenance therapy of the present disclosure is given to a patient in partial remission.

In one embodiment the maintenance therapy of the present disclosure is given to a patient with stable disease.

In one embodiment each dose of the compound of formula (I) is in the range 100 to 900 mg (such as 200, 300, 400, 500, 600, 700 or 800 mg) for example each dose is in the range of 300 to 500 mg, such as 300 mg or 400 mg.

In one embodiment the dose is 200 mg.

In one embodiment the compound of formula (I) or a pharmaceutical composition comprising the same is administered orally.

In one embodiment the compound of formula (I) or a pharmaceutical formulation comprising the same is administered once daily.

In one embodiment the compound of formula (I) or a pharmaceutical formulation comprising the same is administered bi-daily.

In one embodiment the period of cancer remission is prolonged in comparison to a patient population, which does not receive a maintenance therapy.

In one embodiment the remission is full remission.

In one embodiment the remission is partial remission.

In one embodiment the patient's life expectancy is increased in comparison to a patient population, which does not receive a maintenance therapy.

In one embodiment the patient's creatinine levels are monitored during the period of maintenance therapy.

In one embodiment the dose of the compound of formula (I) is reduced in the treatment period, for example to be in the range 100 to 300 mg (such as 200 mg) administered once or twice daily.

In one embodiment the dose reduction is in response to an increase in creatinine levels.

In one embodiment the treatment regimen is one week of receiving compound of formula (I) followed by a week no treatment (of the disclosure).

The compound of formula (I) is surprising well tolerated in patients and has few side effects, which make it particularly suitable for use in maintenance therapy.

Even patients with an apparently bad prognosis may benefit from the maintenance therapy of the present disclosure.

In one embodiment the patient is a mammal, for example a human.

In one embodiment the human patient is a an adult, for example over the age of 18.

In one embodiment the human patient is a child or adolescent, for example under the age of 18.

### DETAILED DISCLOSURE

An enantiomer as employed herein refers to where one enantiomer, for example the R enantiomer or the S enantiomer, in particular the R enantiomer is provided in enantiomeric excess, for example more than 50%, enantiomeric excess, such as 60, 65, 70, 75, 80, 85, 90, 95, 96, 97, 98, 99% enantiomeric excess.

“A pharmaceutical composition comprising the same”, as employed herein refers to a compound of formula (I) including enantiomers, pharmaceutically acceptable salts thereof, Varlitinib or Varlitinib salts formulated with a pharmaceutically acceptable excipient, diluent or carrier.

A therapeutically effective amount as employed herein refers to a dose in the context of treatment or prophylaxis which elicits the desired pharmacological effect.

The compound of formula (I) disclosed herein is a pan-HER inhibitor.

Pan-HER inhibitor as employed herein refers to a molecule that inhibits at two receptors from the ErbB family of proteins, namely ErbB-1 (also known as HER1 and EGFR), ErbB-2 (HER2), ErbB-3 (HER3), ErbB-4(HER4).

In one embodiment the compound of formula (I) at least inhibits the activity of HER1 and HER2, HER1 and HER4 or HER2 and HER4.

In one embodiment the compound of formula (I) at least inhibits the activity of HER1, HER2 and HER4, for example directly inhibits the activity of HER1, HER2 and HER4.

In one embodiment the compound of formula (I) inhibits the activity of HER1, HER2, HER3 and HER4, for example directly inhibits the activity of HER1, HER2, and HER4, and indirectly inhibits the activity of HER3.

Inhibitor as employed refers to the reduction of a relevant biological activity, for example by 5, 10, 15, 20, 25, 30, 35, 40, 45, 50, 55, 60, 65, 70, 75, 80, 85, 90, 95 or 100%, such as when measured in an in vitro assay.

Direct inhibition is where the inhibitor binds directly to or physically blocks a binding interaction to inhibit a biological activity. Thus direct inhibition include steric blocking and allosteric blocking of the receptor.

Indirect inhibition as employed herein refers to where the biological activity in question is inhibited as a result of directly inhibiting a target, other than the indirectly inhibited entity, for example inhibiting by binding to the ligand for the receptor or another mechanism.

Therapy and treatment are employed interchangeably herein unless the context indicates otherwise.

Treatment as employed herein does not necessarily refer to curative treatment.

Cancer maintenance therapy as employed herein is intended to refer to “long term” therapy to prevent or minimise reoccurrence and/or activation of cancer, after first or second line treatment etc to “eliminate” the cancer.

Thus maintenance therapy is therapy intended to prolong cancer remission.

A long-term treatment period is months and years, as opposed to days and weeks.

Cancer remission (also referred to as simply remission) as employed herein includes complete remission and partial remission, unless the context indicated otherwise. Thus even though a cancer has “disappeared” it is not the practice to consider the cancer to be “cured”.

Complete remission (also referred to as full remission) as employed herein is where essentially all the signs of the cancer have disappeared.

Partial remission as employed herein is where some but not all the symptoms of the cancer have disappeared, for example a tumour may be shrunk by therapy or partially removed by surgery and the maintenance therapy keeps the disease stable or prolongs the period over which the disease is stable, such that the tumor does not increase in size, metastasize or similar. Thus partial remission may also be referred to as stable disease.

However, stable disease may also be used to refer to where the cancer has simply stopped progressing but has not necessarily reduced.

In one embodiment the compound of formula (I) is administered as pharmaceutical formulation comprising one or more pharmaceutically acceptable excipients. In one embodiment the compound of formula (I) is administered orally, for example as a tablet or capsule.

Analytical tests for assessing the expression or over expression of HER proteins and their downstream signalling molecules are known and available in the art.

Levels of HER receptor phosphorylation can be measured, for example employing immunohistochemistry or reverse phase protein array and, in particular, an antibody specific to the phosphorylated receptor of interest.

### Cancers

Liver cancer as employed herein refers to cancer of the liver, for example hepatocellular carcinoma including fibrolamellar carcinoma, angiosarcoma and hepatoblastoma.

In one embodiment the liver cancer is hepatocellular carcinoma (HCC).

In one embodiment the cancer is fibrolamellar carcinoma.

Biliary tract cancers as employed herein refers to cholangiocarcinoma, and ampullary carcinoma.

In one embodiment the biliary duct cancer is in a location selected from intrahepatic bile ducts, left hepatic duct, right hepatic duct, common hepatic duct, cystic duct, common bile duct, Ampulla of Vater and combinations thereof.

In one embodiment the biliary duct cancer is in an intrahepatic bile duct.

In one embodiment the biliary duct cancer is in a left hepatic duct.

In one embodiment the biliary duct cancer is in a right hepatic duct.

In one embodiment the biliary duct cancer is in a common hepatic duct.

In one embodiment the biliary duct cancer is in a cystic duct.

In one embodiment the biliary duct cancer is in a common bile duct.

In one embodiment the biliary duct cancer is in an Ampulla of Vater.

In one embodiment the biliary duct cancer is a cancer of the Papilla of Vater.

Cholangiocarcinoma as referred to herein is a form of cancer that is composed of mutated epithelial cells (or cells showing characteristics of epithelial differentiation) that originate in the bile ducts which drain bile from the liver into the small intestine.

General guidelines for operability include:
Absence of lymph node or liver metastases
Absence of involvement of the portal vein
Absence of direct invasion of adjacent organs
Absence of widespread metastatic disease

Gallbladder cancer as employed herein cancer which starts in the gallbladder. The following stages are used for gallbladder cancer:
Stage 0 (carcinoma in situ): Abnormal cells are found in the inner (mucosal) layer of the gallbladder; these abnormal cells may become cancer and spread into nearby normal tissue,
Stage I: Cancer has formed and has spread beyond the inner (mucosal) layer to a layer of tissue with blood vessels or to the muscle layer,
Stage II: Cancer has spread beyond the muscle layer to the connective tissue around the muscle,
Stage IIIA: Cancer has spread through the thin layers of tissue that cover the gallbladder and/or to the liver and/or to one nearby organ (e.g., stomach, small intestine, colon, pancreas, or bile ducts outside the liver),
Stage IIIB: Cancer has spread to nearby lymph nodes and beyond the inner layer of the gallbladder to a layer of tissue with blood vessels or to the muscle layer; or beyond the muscle layer to the connective tissue around the muscle; or through the thin layers of tissue that cover the gallbladder and/or to the liver and/or to one nearby organ,
Stage IVA: Cancer has spread to a main blood vessel of the liver or to 2 or more nearby organs or areas other than the liver. Cancer may have spread to nearby lymph nodes.
Stage IVB: Cancer has spread to either lymph nodes along large arteries in the abdomen and/or near the lower part of the backbone or to organs or areas far away from the gallbladder.

Gastric cancer as employed herein refers to cancer of the stomach, for example squamous cell cancers, lymphoma including non-hodgkin lymphoma, gastrointestinal stromal tumour, or neuroendocrine tumours.

Prostate cancer as employed herein refers to cancer of the prostate, for example ductal adenocarcinoma, transitional cell (urothelial cancer), squamous cell cancer, carcinoid of the prostate, small cell cancer or sarcoma and sarcomatoid cancer.

Pancreatic cancer as employed herein includes exocrine cancers (including rare forms thereof such as cystitic tumours, and cancer of the acinar cells), endocrine pancreatic tumours (including gastrinomas, insulinomas, somatostatinomas, VlPomas, glucagonomas), pancreatoblastoma, sarcomas of the pancreas and lymphoma.

Colorectal cancer as employed herein refers to cancer or the colon and/or rectum and includes squamous cell cancers, carcinoid tumours, sarcomas and lymphomas.

Breast cancer as employed herein refers to cancer of the breast and includes ductal cardinoma in situ, lobular carcinoma in situ, invasive ductal breast cancer, invasive lobular breast cancer, invasive breast cancer, Paget's disease, angiosarcoma of the breast and rare types of breast cancer such as medullary breast cancer, mucinous breast cancer, tubular breast cancer, adenoid cystic carcinoma of the breast metaplastic breast cancer, basal type breast cancer and papillary breast cancer.

Ovarian cancer as employed herein refers to cancer of an ovary and includes endometrioid, clear cell, mucinous, undifferentiated or unclassified, germline and other rare ovarian tumours such as teratoma of the ovary (mature teratoma and immature teratoma) and borderline ovarian tumours. Epithelial ovarian cancers are serious, endometrioid, clear cell, mucinous and undifferentiated or unclassified.

There are more than 30 different types of ovarian cancer which are classified according to the type of cell from which they start. Cancerous ovarian tumours can start from three common cell types:
Surface Epithelium—cells covering the lining of the ovaries
Germ Cells—cells that are destined to form eggs
Stromal Cells—Cells that release hormones and connect the different structures of the ovaries

The present disclosure relates to treatment of ovarian cancer from any source, for example as described herein, in particular epithelium cells. Epithelial ovarian carcinomas (EOCs) account for 85 to 90 percent of all cancers of the ovaries.

Common Epithelial Tumours—Epithelial ovarian tumours develop from the cells that cover the outer surface of the ovary. Most epithelial ovarian tumours are benign (noncancerous). There are several types of benign epithelial tumours, including serous adenomas, mucinous adenomas, and Brenner tumours. Cancerous epithelial tumours are carcinomas—meaning they begin in the tissue that lines the ovaries. These are the most common and most dangerous of all types of ovarian cancers. Unfortunately, almost 70 percent of women with the common epithelial ovarian cancer are not diagnosed until the disease is advanced in stage.

There are some ovarian epithelial tumours whose appearance under the microscope does not clearly identify them as cancerous. These are called borderline tumours or tumours of low malignant potential (LMP tumours). The method of the present disclosure includes treatment of the latter.

Germ Cell Tumours—Ovarian germ cell tumours develop from the cells that produce the ova or eggs. Most germ cell tumours are benign (non-cancerous), although some are cancerous and may be life threatening. The most common germ cell malignancies are maturing teratomas, dysgerminomas, and endodermal sinus tumours. Germ cell malignancies occur most often in teenagers and women in their twenties. Today, 90 percent of patients with ovarian germ cell malignancies can be cured and their fertility preserved.

Stromal Tumours—Ovarian stromal tumours are a rare class of tumours that develop from connective tissue cells that hold the ovary together and those that produce the female hormones, estrogen and progesterone. The most common types are granulosa-theca tumours and Sertoli-Leydig cell tumours. These tumours are quite rare and are usually considered low-grade cancers, with approximately 70 percent presenting as Stage I disease (cancer is limited to one or both ovaries).

Primary Peritoneal Carcinoma—The removal of one's ovaries eliminates the risk for ovarian cancer, but not the risk for a less common cancer called Primary Peritoneal Carcinoma. Primary Peritoneal Carcinoma is closely rated to epithelial ovarian cancer (most common type). It develops in cells from the peritoneum (abdominal lining) and looks the same under a microscope. It is similar in symptoms, spread and treatment.

Stages of Ovarian Cancer Once diagnosed with ovarian cancer, the stage of a tumour can be determined during surgery, when the doctor can tell if the cancer has spread outside the ovaries. There are four stages of ovarian cancer—Stage I (early disease) to Stage IV (advanced disease).

The treatment plan and prognosis (the probable course and outcome of your disease) will be determined by the stage of cancer you have.

Following is a description of the various stages of ovarian cancer:
Stage I—Growth of the cancer is limited to the ovary or ovaries.
Stage IA—Growth is limited to one ovary and the tumour is confined to the inside of the ovary. There is no cancer on the outer surface of the ovary. There are no ascites present containing malignant cells. The capsule is intact.
Stage IB—Growth is limited to both ovaries without any tumour on their outer surfaces. There are no ascites present containing malignant cells. The capsule is intact.
Stage IC—The tumour is classified as either Stage IA or IB and one or more of the following are present: (1) tumour is present on the outer surface of one or both ovaries; (2) the capsule has ruptured; and (3) there are ascites containing malignant cells or with positive peritoneal washings.
Stage II—Growth of the cancer involves one or both ovaries with pelvic extension.
Stage IIA—The cancer has extended to and/or involves the uterus or the fallopian tubes, or both.
Stage IIB—The cancer has extended to other pelvic organs.
Stage IIC—The tumour is classified as either Stage IIA or IIB and one or more of the following are present: (1) tumour is present on the outer surface of one or both ovaries; (2) the capsule has ruptured; and (3) there are ascites containing malignant cells or with positive peritoneal washings.
Stage III—Growth of the cancer involves one or both ovaries, and one or both of the following are present: (1) the cancer has spread beyond the pelvis to the lining of the abdomen; and (2) the cancer has spread to lymph nodes. The tumour is limited to the true pelvis but with histologically proven malignant extension to the small bowel or omentum.
Stage IIIA—During the staging operation, the practitioner can see cancer involving one or both of the ovaries, but no cancer is grossly visible in the abdomen and it has not spread to lymph nodes. However, when biopsies are checked under a microscope, very small deposits of cancer are found in the abdominal peritoneal surfaces.
Stage IIIB—The tumour is in one or both ovaries, and deposits of cancer are present in the abdomen that are large enough for the surgeon to see but not exceeding 2 cm in diameter. The cancer has not spread to the lymph nodes.
Stage IIIC—The tumour is in one or both ovaries, and one or both of the following is present: (1) the cancer has spread to lymph nodes; and/or (2) the deposits of cancer exceed 2 cm in diameter and are found in the abdomen.
Stage IV—This is the most advanced stage of ovarian cancer. Growth of the cancer involves one or both ovaries and distant metastases (spread of the cancer to organs located outside of the peritoneal cavity) have occurred. Finding ovarian cancer cells in pleural fluid (from the cavity which surrounds the lungs) is also evidence of stage IV disease.

In one embodiment the ovarian cancer is: type I, for example IA, IB or IC; type II, for example IIA, IIB or IIC; type III, for example IIIA, IIIB or IIIC; or type IV. The present disclosure relates to treatment of any stage of ovarian cancer, in particular as described herein.

Lung cancers are classified according to histological type and are categorized by the size and appearance of the malignant cells seen by a histopathologist under a microscope. For therapeutic purpose, two broad classes are distinguished: non-small cell lung carcinoma and small cell lung carcinoma.

In one embodiment the epithelial cancer is lung cancer, for example small-cell selected from lung cancer (SCLC) and non-small-cell lung cancer (NSCLC).

Non-small-cell lung carcinoma—The three main subtypes of NSCLC are adenocarcinoma, squamous-cell carcinoma and large-cell carcinoma.

Nearly 40% of lung cancers are adenocarcinoma, which usually originates in peripheral lung tissue. A subtype of adenocarcinoma, the bronchioloalveolar carcinoma, is more common in female never-smokers, and may have a better long term survival.
Squamous-cell carcinoma accounts for about 30% of lung cancers. They typically occur close to large airways. A hollow cavity and associated cell death are commonly found at the center of the tumour. About 9% of lung cancers are large-cell carcinoma. These are so named because the cancer cells are large, with excess cytoplasm, large nuclei and conspicuous nucleoli.

Small-cell lung carcinoma—In small-cell lung carcinoma (SCLC), the cells contain dense neurosecretory granules (vesicles containing neuroendocrine hormones), which give this tumour an endocrine/paraneoplastic syndrome association. Most cases arise in the larger airways (primary and secondary bronchi). These cancers grow quickly and spread early in the course of the disease. Sixty to seventy percent have metastatic disease at presentation.

In one embodiment the cancer is non-small lung carcinoma.

In one embodiment there is provided treatment of renal cancer, for example renal cell carcinoma and/or urothelial cell carcinoma using an oncolytic adenovirus as disclosed herein. Other examples of renal cancer include squamous cell carcinoma, juxtaglomerular cell tumour (reninoma), angiomyolipoma, renal oncocytoma, Bellini duct carcinoma, clear-cell sarcoma of the kidney, mesoblastic nephroma, Wilms' tumour, mixed epithelial stromal tumour, clear cell adenocarcinoma, transitional cell carcinoma, inverted papilloma, renal lymphoma, teratoma, carcinosarcoma, and carcinoid tumour of the renal pelvis.

In one embodiment the cancer is bladder cancer, for example is any of several types of malignancy arising from the epithelial lining (i.e., the urothelium) of the urinary bladder. About 90% of bladder cancers are transitional cell carcinoma. The other 10% are squamous cell carcinoma, adenocarcinoma, sarcoma, small cell carcinoma, and secondary deposits from cancers elsewhere in the body. The staging of is given below.

T (Primary tumour)
TX Primary tumour cannot be assessed
TO No evidence of primary tumour
Ta Non-invasive papillary carcinoma
Tis Carcinoma in situ (Tat tumour′)
T1 Tumour invades sub epithelial connective tissue
T2a Tumour invades superficial muscle (inner half)
T2b Tumour invades deep muscle (outer half)
T3 Tumour invades perivesical tissue:
T3a Microscopically
T3b Macroscopically (extravesical mass)
T4a Tumour invades prostate, uterus or vagina
T4b Tumour invades pelvic wall or abdominal wall

N (Lymph nodes)
NX Regional lymph nodes cannot be assessed
N0 No regional lymph node metastasis
N1 Metastasis in a single lymph node 2 cm or less in greatest dimension
N2 Metastasis in a single lymph node more than 2 cm but not more than 5 cm in greatest dimension, or multiple lymph nodes, none more than 5 cm in greatest dimension
N3 Metastasis in a lymph node more than 5 cm in greatest dimension M (Distant metastasis)
MX Distant metastasis cannot be assessed
M0 No distant metastasis
M1 Distant metastasis.

Thyroid cancer as employed herein refers to cancer of the thyroid originating from follicular or parafollicular thyroid cells and includes papillary thyroid cancer (75% to 85% of cases); follicular thyroid cancer (10% to 20% of cases); medullary thyroid cancer (5% to 8% of cases)—cancer of the parafollicular cells, often part of multiple endocrine neoplasia type 2; poorly differentiated thyroid cancer; anaplastic thyroid cancer (less than 5% of cases) is not responsive to treatment and can cause pressure symptoms, thyroid lymphoma, squamous cell thyroid carcinoma, sarcoma of thyroid.

Renal cancer as employed herein refers to cancer of the kidney, for example renal cell carcinoma and transitional cell carcinoma of the renal pelvis, such as squamous cell carcinoma, juxtaglomerular cell tumor (reninoma), angiomyolipoma, renal oncocytoma, bellini duct carcinoma, clear-cell sarcoma of the kidney, mesoblastic nephroma, Wilms' tumor, mixed epithelial stromal tumor, clear cell adenocarcinoma, transitional cell carcinoma, inverted papilloma, renal lymphoma, teratoma, carcinosarcoma; carcinoid tumor of the renal pelvis.

Bladder cancer as employed herein refers to cancer of the bladder including transitional cell bladder cancer, carcinoma in situ, papillary cancer and rarer types of bladder cancer such as squamous cell cancer and adenocarcinoma.

Oesphageal cancer as employed herein refers to cancer of the oesphagus including esophageal squamous-cell carcinomas, esophageal adenocarcinomas, and variants of squamous-cell carcinoma, and non-epithelial tumors, such as leiomyosarcoma, malignant melanoma, rhabdomyosarcoma, lymphoma, among others.
Head and neck cancer as employed herein refers to cancer of the neck and/or head, including mouth cancer, nasopharyngeal cancer, oropharyngeal cancer, paranasal sinus cancer and salivary gland cancer.

Cervical cancer as employed herein refers to cancer of the cervix, including squamous cell cancer, adenocarcinoma and lymphoma, in particular squamous cell cancer and adenocarcinoma.

“Comprising” in the context of the present specification is intended to mean “including”. Where technically appropriate, embodiments of the invention may be combined.

Embodiments are described herein as comprising certain features/elements. The disclosure also extends to separate embodiments consisting or consisting essentially of said features/elements.

Embodiments generally describe one technical aspect in detail. The disclosure herein includes the combination of 1, 2, more embodiments as technically appropriate.

Technical references such as patents and applications are incorporated herein by reference.

Any embodiments specifically and explicitly recited herein may form the basis of a disclaimer either alone or in combination with one or more further embodiments.

The invention will now be described with reference to the following examples, which are merely illustrative and should not in any way be construed as limiting the scope of the present invention.

### EXAMPLES

### Example 1

A female patient diagnosed with squamous cell carcinoma of the cervix was initially treated with radiation therapy and concurrent weekly cisplatin. She had recurring disease the following year. She subsequently received a number of lines of chemotherapy, including topotecan/cisplating and ifosfamide. The cancer continued to progress.

She received 300 mg bi-daily of varlitinib for 6.5 years.

The patient received varlitinib maintenance therapy for about 6.5 years before developing transitional cell carcinoma of the bladder. It is likely that this bladder carcinoma was a result receiving pelvic radiation and/or treatment with ifsoamide.

### Example 2

A 69 year old female with choloangiocarcinoma (CCA), namely stage VI adenocarcinoma had already received surgery, was administered varlitinib at a dose of 400 mg bi-daily in combination with doublet chemotherapy for six 28-day cycles. Varlitinib was then used as maintenance therapy as monotherapy for 10 additional cycles, of 28 days of treatment.

The monotherapy dose was reduced from 400 mg bi-daily to 300 mg bi-daily due to an increase in G3 creatinine levels observed in the patient at cycle 10 day 15.

In the period of 10 cycles of maintenance therapy, no target lesion was observed and the patient remained in stable disease.

### Example 3

A 51 year old Asian (Chinese) female (ASLAN001-002SG LST/0013) had the past history of meningioma post left craniotomy for tumor excision in 2007. She was diagnosed with intrahepatic cholangiocarcinoma in July 2013, and received left hemihepatectomy followed by adjuvant chemotherapy (gemcitabine plus cisplatin) from 14 Aug. 2013 to 8 Jan. 2014. Disease relapse was found in 15 Jul. 2015. Thus, she received systemic treatment with gemcitabine plus cisplatin from 13th May to 1 Jul. 2015.

She joined the clinical trial, ASLAN001-002SG. The study treatment of bi-weekly FOLFOX plus continuous 400 mg varlitinib per oral twice a day started on 4 Aug. 2015. Due to grade 3 hyperbilirubinemia which occurred on 8 Aug. 2015, varlitinib was interrupted and reduced to 300 mg twice a day in combination with FOLFOX. After 9 cycles (14 days/cycle) of combined regimen, she continues varlitinib monotherapy. To date, she receives varlitinib maintenance treatment for 504 days, and her tumor is still in partial response status.

### Example 4

A 68 year old Asian (Chinese) female (ASLAN001-002 2A101) had the past history of type 2 diabetes mellitus and hypertension for more than 10 years. She was diagnosed with stage IV cholangiocarcinoma in November 2014.

She joined the clinical trial, ASLAN001-002. The study treatment of cisplatin/5-FU plus continuous 400 mg varlitinib per oral twice a day started on 27 Jan. 2015.

After 6 cycles (28 days/cycle) of combined regimen, she continued 400 mg varlitinib monotherapy for another 99 days. Due to creatinine increase, the dosage of varlitinib was tapered to 300 mg twice a day 1 week on and 1 week off. The total treatment duration of varlitinib maintenance therapy was 287 days. Her best tumor response was non-complete response/non-progressive disease (non-target disease).

The study treatment was discontinued due to disease progression.

### Example 5

A 64 year old Asian (Chinese) male (ASLAN001-002 2A102) had the past history of hypertension for more than 5 years. He was diagnosed with stage IV gastric cancer in April 2015.

He joined the clinical trial, ASLAN001-002. The study treatment of cisplatin/5-FUplus continuous 400 mg varlitinib per oral twice a day started on 12 May 2015. After 6 cycles (28 days/cycle) of combined regimen, he continued 400 mg varlitinib monotherapy for another 112 days. Due to creatinine increase, the dosage of varlitinib was tapered to 300 mg twice a day. The total treatment duration of varlitinib maintenance therapy was 243 days. His best tumor response was partial response. The study treatment was discontinued due to disease progression.

### Example 6

A 63 year old Caucasian male (ASLAN001-002SG DNT/0012) had the past history of type 2 diabetes mellitus, atrial fibrillation, hypertension, hyperlipidemia, and grade 1 hearing loss. He was diagnosed with stage IV bladder cancer and received radical cystoprostatectomy on 13 Dec. 2013. He also received cisplatin based concurrent radiotherapy on 20 Feb. 2014, and gemcitabine plus cisplatin from 10 Apr. to 8 May 2015.

He joined the clinical trial, ASLAN001-002SG. The study treatment of bi-weekly FOLFOX plus continuous 300 mg varlitinib per oral twice a day started on 24 Aug. 2015. After 9 cycles (14 days/cycle) of combined regimen, he continued varlitinib monotherapy. The total treatment duration of varlitinib maintenance therapy was 140 days. His best tumor response was stable disease. The study treatment was discontinued due to disease progression.

### Example 7

A 57 year old Asian (Chinese) female (ASLAN001-002 2B302) had the underlying disease of gastro-esophageal reflux disease. She was diagnosed with stage IV gastric cancer in Jul. 2016.

She joined the clinical trial, ASLAN001-002. The study treatment of cisplatin/capecitabine plus continuous 300 mg varlitinib per oral twice a day started on 6 Sep. 2016. After 6 cycles (21 days/cycle) of combined regimen, she continues varlitinib monotherapy. The total treatment duration of varlitinib maintenance therapy was 134 days. Her best tumor response was stable disease. The study treatment was discontinued due to disease progression.

1.-21. (canceled)

22. A method of cancer maintenance therapy comprising administering a therapeutically effective amount of a compound of formula (I): 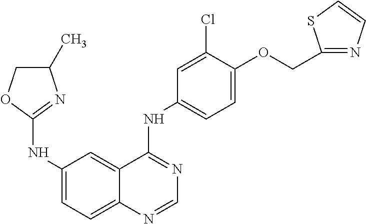 [SMILES: CC1COC(Nc2ccc3ncnc(Nc4ccc(OCc5nccs5)c(Cl)c4)c3c2)=N1]
an enantiomer thereof or pharmaceutically acceptable salt of any one of the same, wherein the therapy is continued for 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 months or more.

23. A method according to claim 22, wherein the compound of formula (I) is: 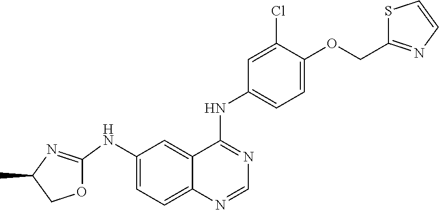 [SMILES: C[C@@H]1COC(Nc2ccc3ncnc(Nc4ccc(OCc5nccs5)c(Cl)c4)c3c2)=N1]
or a pharmaceutically acceptable salt thereof.

24. A method according to claim 22, wherein the compound of formula (I) is provided as the free base.

25. A method according to claim 22, wherein the cancer is an epithelial cancer.

26. A method according to claim 22, wherein the cancer is selected from the group comprising liver cancer, biliary tract cancer, breast cancer (such as none ER+breast cancer), prostate cancer, colorectal cancer, ovarian cancer, cervical cancer, lung cancer, gastric cancer, pancreatic cancer, gallbladder cancer, bone cancer, bladder cancer, head and neck cancer, thyroid cancer, skin cancer, renal cancer, and oesophagus cancer.

27. A method according to claim 26, wherein the cancer is selected from gastric cancer, hepatocellular carcinoma and cholangiocarcinoma.

28. A method according to claim 22, wherein the cancer is HER2 positive or HER2 amplified.

29. A method according to claim 22, wherein each dose of the compound of formula (I) or composition comprising the same is in the range 100 to 900 mg.

30. A method according to claim 29, wherein each dose is in the range of 300 to 500 mg, such as 400 mg.

31. A method according to claim 22, wherein the compound of formula (I) or a pharmaceutical composition comprising the same is administered orally.

32. A method according to claim 22, wherein the compound of formula (I) or a pharmaceutical formulation thereof is administered bi-daily.

33. A method according to claim 22, wherein the period of cancer remission is prolonged in comparison to a patient population which do not receive a maintenance therapy.

34. A method according to claim 33, wherein the remission is full remission.

35. A method according to claim 33, wherein the remission is partial remission.

36. A method according to claim 22, wherein the patient's life expectancy is increased in comparison to a patient population which do not receive a maintenance therapy.

37. A method according to claim 22, wherein the patient's creatinine levels (in particular G3 creatinine levels) are monitored during the period of maintenance therapy.

38. A method according to claim 22, wherein the dose of the compound of formula (I) is reduced in the treatment period, for example to be in the range 100 to 300 mg administered once or twice daily.

39. A method according to claim 38, wherein the dose reduction is in response to an increase in creatinine levels (in particular an increase in Ge creatinine levels).
